(19) United States
(12) Patent Application Publication
(10) Pub. No.: US 2020/0281842 A1
(43) Pub. Date: Sep. 10, 2020

(54) METHOD FOR SOLVENT-FREE SKIN CARE PRODUCTS

(71) Applicant: Reza Fatahi, Encino, CA (US)

(72) Inventor: Reza Fatahi, Encino, CA (US)

(21) Appl. No.: 16/294,852

(22) Filed: Mar. 6, 2019

(51) Int. Cl. A61K 8/86; A61Q 19/00; A61K 8/03

(52) U.S. Cl. CPC A61K 8/86; A61Q 19/00; A61K 2800/80; A61K 2800/30; A61K 2800/52; A61K 8/03

(57) ABSTRACT

We report a method of manufacturing solvent-free skin care products by substituting volatile organic compounds for polysorbate surfactants. A minimum effective concentration of food-grade polysorbate surfactants resulted in a fragrance composition that naturally separates layers following homogenization with gentle mixing.

RELATED TO application Ser. No. 16/270,606

### BACKGROUND

### Field of the Invention

This invention concerns novel liquid skin care compounds and their method of production.

### Background of Invention

Traditional Organic Solvents (TOS) are chemicals such as alcohol, acetone, ethers, and chloroform that are used in fragrances to mix oil and aqueous phases to a single layer.

TOS are generally toxic substances that may be irritating to sensitive mucosal tissues including the skin, eyes, olfaction, and inner lining of the respiratory system.

Further, TOS have been associated with denaturing the active state of vitamins and nutrients that may be added to fragrances for skin health.

TOS are generally carcinogenic and harsh chemicals. Thus, it is desirable to have a method of manufacturing skin care products that do not contain TOS.

### SUMMARY OF INVENTION

The method of this invention is centered around the use of polysorbate surfactants as a replacement for organic solvents or alcohol in skin care compounds. The invention results in a liquid product with separate oil and water phases that homogenize following gentle shaking.

The invention is the minimum effective polysorbate surfactant concentration to homogenize oil and water layers. Polysorbate surfactants are a type of nonionic surfactants, and surfactants are soap-like substances whose chemical structure is similar to fats. They do not evaporate at room temperature as do solvents.

The minimum concentration of commercially available food-grade polysorbate surfactants were measured to be similar. The minimum concentration ranged as low as 2% total-oil-wt and always less than 5% total-oil-wt.

### BRIEF DESCRIPTION OF DRAWINGS

FIG. 1: shows the natural separation of oil and aqueous layers in a fragrance solution without solvents and without surfactants in a settled state. The oil layer is demarcated with dashed lines.

FIG. 2: shows the natural mixture of oil in water in a fragrance solution without solvents and without surfactants after mechanical stirring.

FIG. 3: shows the natural separation of oil and aqueous layers in a fragrance solution with the minimal effective concentration of polysorbate surfactants in a settled state. The oil layer is demarcated with dashed lines.

FIG. 4: shows the homogeneous mixture of oil and aqueous layers in a fragrance solution with the minimal effective concentration of polysorbate surfactants after mechanical stirring.

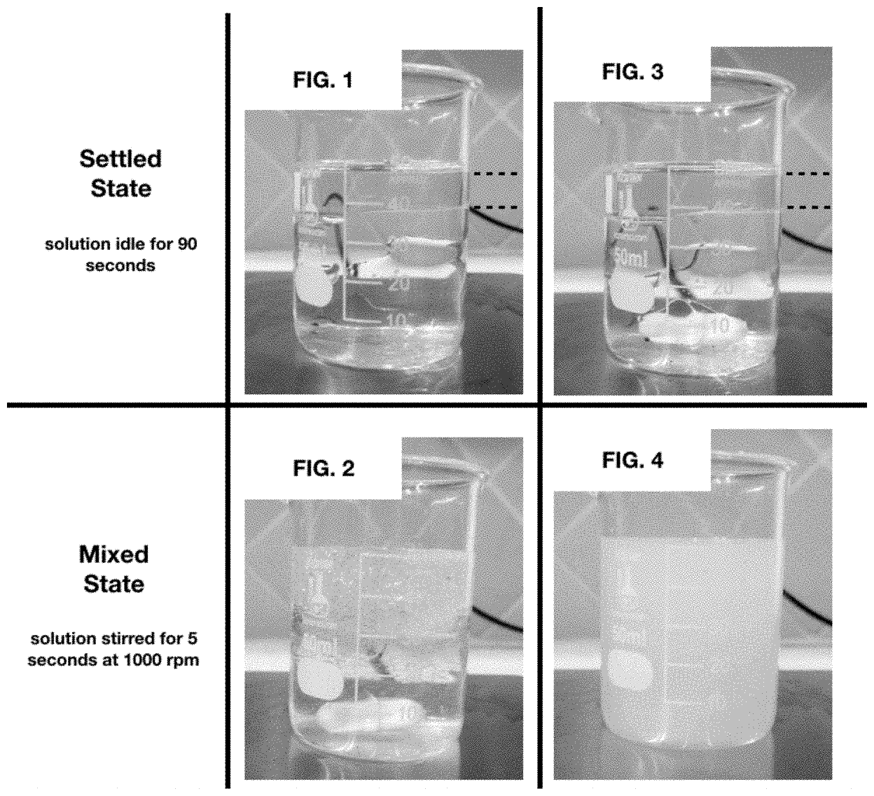

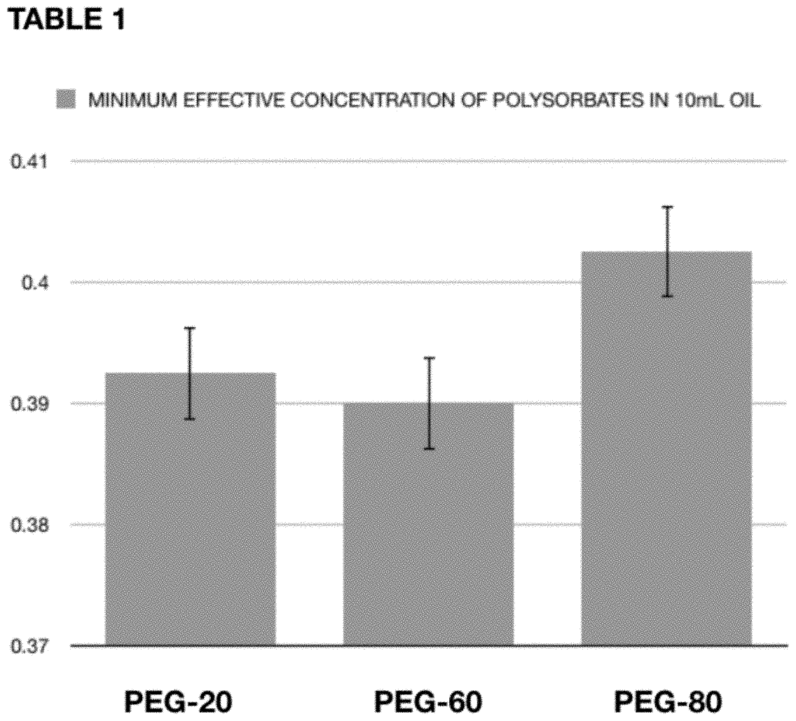

TABLE 1: shows the minimum effective concentration of various polysorbate surfactants in a 20% oil formulation at 50 mL total volume to completely homogenize oil and water layers, while concentrations below these were not found to fully homogenize water and oil layers.

### DETAILED DESCRIPTION OF INVENTION

Oil and water do not mix unless there is an agent to facilitate their mixture. Organic solvents are widely utilized in the manufacturing of skin care products to blend oil and aqueous layers in to one homogeneous liquid layer. However, these solvents have also been found to have detrimental effects against the bioactive state of nutrients.

It is desirable for oil and water to homogenize in order to evenly disperse fragrant components in a liquid skin care product, and to be low enough in viscosity to conveniently dispense with standard atomizers and sprays. Also, the applicant of this product should not feel oily as would be the case with natural oil products.

An “organic solvent” is a type of volatile organic compound (VOC). VOCs are organic chemicals that can evaporate at room temperature. Classifications of organic solvents include chlorinated solvents, allopathic hydrocarbons, alcohols, glycols, esters, ethers, and aldehydes. Specific examples of organic solvents include alcohol, chloroform, and acetone. Almost every organic solvent is health hazard.

In addition to their general toxic characteristic, VOCs have been found to denature vitamins, coenzymes, and enzyme proteins.

The present invention provides a skin care composition that eliminates the use of VOCs by substituting them with polysorbate surfactants to homogenize oil and aqueous layers.

Polysorbate surfactants are a type of anionic surfactant. They behave like soap, and mix water and oil. High concentrations of polysorbate surfactants may result in soap bubbles or suds. Here, we were interested to utilize the minimum concentration of polysorbate surfactants to mix oil and water layers of nutriated skin care compounds.

The stability and relative nontoxicity of polysorbate surfactants allows them to be used as detergents and emulsifiers in a number of domestic, scientific, and pharmacological applications. They are also found in food products, hence, they may be referred as “food-grade”.

A minimum concentration of polysorbate surfactant was defined by volumetric titration into various fragrance composition mixtures ranging from 10-50% oil in water on a stirring plate with continuous stirring. The results were similar across polysorbate types and found to be as low as 2% total-oil-wt of compounds, and always below 5% total-oil-wt concentration.

The amount of time required for the oil and water layers to separate for various concentrations of polysorbate surfactants was measured. These were determined to be as low as 30 seconds for the lowest concentrations and as high as one minute or more for higher concentrations. All of the concentration ranges were found to result in a separation of oil and water layers once the compound is allowed to settle.

The object of the present invention is a minimum polysorbate surfactant concentration to substitute the use of VOCs. Our concentration was found to be effective to homogenize oil and aqueous layers following gentle shaking. For our purposes, the natural separation of oil and aqueous layers is desired as a visual indicator that VOCs are not used.

The homogenized solutions also displayed favorable viscosity characteristics for the dispersion of the liquid fragrance product with standard atomizers and sprays. With this system, the fragrance in it's settled state will have a separation of the oil layer from the aqueous layers, thus requiring the user to mix layers with gentle shaking prior to the application of the fragrance to their body or skin. Further, since there are no VOCs this product may be used on animals and in clinical settings.

The absence of VOCs and organic solvents provides a suitable environment for the addition of nutrients to the fragrance composition, including water soluble vitamins, oil soluble vitamins, and coenzymes. The absence of VOCs eliminates any damage to the bioactive properties of nutrients caused by solvents.

The present invention can therefore result in a liquid skin care product that can be applied through conventional atomizers and sprayers, that promotes nutrient availability in their active state, and avoids irritation to the skin, olfaction, and respiratory systems due to the absence of VOCs in the composition.

1. A method for preparing liquid skin care products without organic solvents.

2. A method for preparing liquid skin care products with food-grade polysorbate surfactants including PEG-20, PEG-60, and PEG-80.

3. A method for preparing liquid skin care products with any liquid polysorbate surfactant types.

4. A method according to claims 2 and 3 at their minimal concentrations.

5. A method according to claim 4 at a concentration as low as 2% total-oil-wt and always below 5% total-oil-wt of the final composition.

6. A method according to claim 5 wherein any lower concentration would not homogenize water and oil layers following gentle shaking.

7. A method according to claims 1-6 of manufacturing solvent-free liquid skin care products.

8. A method according to claim 7 wherein solvents are not used and do not exist.

9. A method according to claim 8 that eliminates the denaturing of bioactive nutrients caused by solvents.

10. A method according to claims 8 that eliminates the denaturing of essential oil benefits and mineral nanostructures caused by solvents.

11. A method according to claims 7 and 8 wherein the composition is a liquid product.

12. A liquid skin care product according to claims 7-11 wherein added nutrients have no loss of bioactive potency caused by the presence of organic solvents.

13. A liquid skin care product according to claims 7-11 wherein added nutrients have no loss of essential oil and mineral benefits caused by the presence of organic solvents.

14. A liquid skin care product according to claim 11 that dispenses with standard atomizers and sprays.

15. A liquid skin care product according to claim 11 that always forms a bilayer (i.e. separation of oil and water layers) after allowing the solution time to settle.

16. A liquid skin care product according to claim 11 that fully homogenizes following gentle shaking.

17. A liquid skin care product according to claim 11 that would not homogenize with lower concentrations according to claim 6.

18. A liquid skin care product according to claims 1-17 that behave similarly across different food-grade polysorbate surfactant types including PEG-20, PEG-60, and PEG-80.

19. A liquid skin care product according to claims 1-18 that does not use any other products to homogenize oil and water layers.

20. A liquid skin care product according to claims 1-19 that naturally separates oil and water layers 90 seconds or less after allowing the solution time to settle.
